SEQUENCE LISTING (32 sequences)

SEQ ID NO: 1 - DNA, 20 bp, Escherichia coli
cggctttatatttacccagc

SEQ ID NO: 2 - DNA, 18 bp, Escherichia coli
cgcaacaaacgcggctac

SEQ ID NO: 3 - DNA, 20 bp, Escherichia coli
acgcttcaaccttcataccg

SEQ ID NO: 4 - DNA, 20 bp, Escherichia coli
gatgccgtttcgctcatagt

SEQ ID NO: 5 - DNA, 20 bp, Escherichia coli
atggatatcgtcgggtttga

SEQ ID NO: 6 - DNA, 20 bp, Escherichia coli
aagcaataacgttccggttg

SEQ ID NO: 7 - DNA, 20 bp, Escherichia coli
gttcgcctgcttcgttagtc

SEQ ID NO: 8 - DNA, 20 bp, Escherichia coli
ctgccgctatgttgaagaca

SEQ ID NO: 9 - DNA, 20 bp, Escherichia coli
aaagcgatgctgaaaggtgt

SEQ ID NO: 10 - DNA, 20 bp, Escherichia coli
agaaagcgtcaggcagtgtt

SEQ ID NO: 11 - DNA, 20 bp, Escherichia coli
gcgccaattgactcttgaat

SEQ ID NO: 12 - DNA, 20 bp, Escherichia coli
cctgccagcagagtagaacc

SEQ ID NO: 13 - DNA, 16 bp, Escherichia coli
cgcgtaacaattgtgg

SEQ ID NO: 14 - DNA, 16 bp, Escherichia coli
ctggattttttccagc

SEQ ID NO: 15 - DNA, 25 bp, Escherichia coli
ctgtttgatgccgtctaactcgttc

SEQ ID NO: 16 - DNA, 25 bp, Escherichia coli
ctttatccatgagtcgccgcctgtg

SEQ ID NO: 17 - DNA, 20 bp, Escherichia coli
tacccaacgaaccaattttc

SEQ ID NO: 18 - DNA, 20 bp, Escherichia coli
gctggaagagctgaaaaagc

SEQ ID NO: 19 - DNA, 20 bp, Escherichia coli
ttacgtgccattgcggagtg

SEQ ID NO: 20 - DNA, 20 bp, Escherichia coli
tcacgatacagtagcgggtg

SEQ ID NO: 21 - DNA, 20 bp, Escherichia coli
ccaccagctttcatctccat

SEQ ID NO: 22 - DNA, 20 bp, Escherichia coli
tattccctccagcgaaattg

SEQ ID NO: 23 - DNA, 20 bp, Escherichia coli
tcagatccgggaagatgaac

SEQ ID NO: 24 - DNA, 20 bp, Escherichia coli
tgtgctgatggaagagatcg

SEQ ID NO: 25 - DNA, 20 bp, Escherichia coli
tgaatgcagtctgcttggtc

SEQ ID NO: 26 - DNA, 20 bp, Escherichia coli
aaaacgttggtccgaaacac

SEQ ID NO: 27 - DNA, 20 bp, Escherichia coli
caccgtactggttgacgatg

SEQ ID NO: 28 - DNA, 20 bp, Escherichia coli
cacaacgcctttgctcagta

SEQ ID NO: 29 - DNA, 20 bp, Escherichia coli
tctacccatttccaggcttc

SEQ ID NO: 30 - DNA, 20 bp, Escherichia coli
tgggacaccctgagtgcacg

SEQ ID NO: 31 - DNA, 20 bp, Escherichia coli
gagctgtccggcatacggtc

SEQ ID NO: 32 - DNA, 20 bp, Escherichia coli
cgctggcaggcaaaccggtg

(19) United States
(12) Patent Application Publication
(10) Pub. No.: US 2009/0253164 A1
(43) Pub. Date: Oct. 8, 2009

(54) E. COLI FOR EFFICIENT PRODUCTION OF CARATENOIDS

(76) Inventors: Pornkamol Unrean, Minneapolis, MN (US); Cong T. Trinh, Saint Paul, MN (US); Friedrich Srienc, Lake Elmo, MN (US)

Correspondence Address: DARDI & ASSOCIATES, PLLC, 220 S. 6TH ST., SUITE 2000, U.S. BANK PLAZA, MINNEAPOLIS, MN, 55402, US

(21) Appl. No.: 12/324,373

(22) Filed: Nov. 26, 2008

Related U.S. Application Data
(60) Provisional application No. 60/990,826, filed on Nov. 28, 2007.

(51) Int. Cl. C12P 23/00; C12N 1/21; C12N 15/87

(52) U.S. Cl. 435/67; 435/252.33; 435/471

(57) ABSTRACT

An improved E. coli for carotenoid production comprising exogenous nucleic acids for expressing a carotenoid that the E. coli requires as necessary condition for the E. coli to reproduce. Some E. coli embodiments have diminished or abrogated expression of a gene in the group consisting of ldhA, frdA, poxB, pta, adhE, pykF, zwf, and maeB.

### CROSS-REFERENCE TO RELATED PATENT APPLICATIONS

This patent application claims priority to U.S. Ser. No. 60/990,826 filed Nov. 28, 2007 which is hereby incorporated by reference herein.

### GOVERNMENT SUPPORT

The United States government may have rights to these inventions.

### BACKGROUND

Carotenoids are natural color pigments having long, unsaturated hydrocarbon chains of about 30 to about 50 carbons that are synthesized in certain plants and microorganisms. Most of the commercially important carotenoids are currently produced by chemical synthesis or by extraction from natural sources such as plants. These production processes are limited in terms of quantity as well as structural diversity of carotenoids. Carotenoids can have a variety of functions. They are the light-harvesting pigments in the photosynthetic process, UV-protective compounds, regulators of membrane fluidity and antioxidant materials to protect cells against harmful oxygen radicals. Recently, it has been found that carotenoids play an important role in the prevention of cancer and of certain chronic degenerative diseases including heart disease, immune deficiency and aging (23). Carotenoids are currently used as nutrient supplements, pharmaceuticals and food colorants. To date, more than 600 carotenoids have been characterized. Most of them exist as biosynthetic intermediates. Therefore, they occur only in trace amounts in natural sources and are difficult to extract in sufficient amounts to be useful for applications. Therefore, carotenoid production has been attempted using microbial fermentation. Carotenogenic recombinant E. coli have been engineered for expressing various carotenoids including zeaxanthin, lycopene and β-carotene (1, 16). However, carotenoid production levels in recombinant E. coli are still not high enough for an economical large scale production.

### SUMMARY

Inefficient carotenoid biosynthesis pathways were eliminated to create E. coli strains that produce carotenoids with high efficiency in a wide variety of culture environments. The E. coli can be made to not only produce carotenoids efficiently and at yields that are greater than control strains but also to actually require the carotenoids as a condition for the cells to reproduce. Thus unwanted mutants that do not produce the carotenoids will tend to be eliminated during culture to thereby promote a stable and robust production. Further, E. coli can be made to have only metabolic pathways that lead to carotenoid production, thus minimizing unwanted by products. Some strains had a combination of eight specific gene deletions that contributed to efficiency.

An embodiment is an E. coli for carotenoid production comprising exogenous nucleic acids for expressing a carotenoid and diminished or abrogated expression of one or more, or any combination of, a gene in the group consisting of ldhA, frdA, poxB, pta, adhE, pykf, zwf, and maeB. The carotenoid produced may comprise diapolycopendial (DPL) and/or diapolycopendioic acid (DPA). The E. coli may be made to require a carotenoid as a necessary condition for the E. coli to reproduce. The E coli may be made to be free of metabolic pathways that (i) receive glucose as an input and (ii) do not result in production of the carotenoid. The E. coli may have all of its metabolic pathways that receive glucose as an input also produce carotenoid as an output; some of these pathways may also be necessary for growth. Some embodiments of E. coli have only 1 to about 10 metabolic pathways to produce a carotenoid, e.g., only about 5 such pathways. The E. coli may be cultured and carotenoids recovered. E. coli may be modified to produce a higher amount or yield of a carotenoid compared to a parent strain or wild-type strain.

### BRIEF DESCRIPTION OF THE FIGURES

FIG. 1. Metabolic network of recombinant E. coli expressing biosynthesis of carotenoids, diapolycopendial/diapolycopendioic acid. Reaction designations in the network are corresponded to the reaction number in Elementary Mode Analysis. Reactions shown in circles (designated R57 or R58) are introduced into the cell via plasmid, pACMNOx. External metabolites are underlined.

FIG. 2. Effect of single deleted gene on synthesis of diapolycopendioic acid, biomass formation and fractional remaining modes. Maximum yield of carotenoids () and biomass (▴) as well as fractional remaining mode (▪) of a single gene deletion are shown. The yield is defined as carbon mole per carbon mole ratio of product to glucose substrate. Fractional remaining mode is determined as the ratio of remaining elementary modes in the single gene knockout to those in the wild-type. Potential knockout target is the gene or genes in which the deletion of reaction or reactions specific to these genes still maintain maximum yield of carotenoid and biomass while minimize fractional remaining modes.

FIG. 3. Elementary modes of CRT028/pACMNOx as compared with total available elementary modes existing in wild-type MG1655/pACMNOx. All elementary modes available in the wild-type is shown in ◯. Elementary modes remained after multiple gene knockouts in the mutant is shown in (with arrows labeled e1-e5, 4 moles NADP per mole DPA assumed) and in ▴ (8 moles NADP per mole DPA assumed). An experimental yield of CRT028/pACMNOx is shown in ▪ (also labeled). Dash line connecting the highest-yielding carotenoid mode with the highest-yielding biomass mode represented the yield performance of linear combinations of the carotenoid and biomass highest-yielding pathways. The negative slope revealed a connected and inverse relationship between carotenoid and biomass formation.

FIG. 4A is a depiction of the detailed pathway of elementary mode No. 1 in multiple-genes knockout CRT028/pACMNOx.

FIG. 4B is a depiction of the detailed of elementary mode No. 2 in multiple-genes knockout CRT028/pACMNOx.

FIG. 4C is a depiction of the detailed pathway of elementary mode No. 3 in multiple-genes knockout CRT028/pACMNOx.

FIG. 4D is a depiction of the detailed pathway of elementary mode No. 4 in multiple-genes knockout CRT028/pACMNOx.

FIG. 5. Effect of number of NADP cofactor on number of available elementary modes (▪) and minimum predicted yield of diapolycopendioic acid () in CRT028/pACMNOx

FIG. 6. Absorption spectra of extract solutions using acetone (a) and 10% KOH (b) from E. coli cells expressing carotenoid plasmid pACMNOx. Maximum absorbance was at 506 nm in the acetone extract and at 490 nm in the KOH extract. The wavelength of maximum absorbance in both extracts were in agreement with those reported in Tao et al. (2005) for diapolycopendial in acetone and diapolycopendioic acid in 10% KOH solution respectively.

FIG. 7. Genes deletion in mutant CRT028 confirmed by PCR and gel electrophoresis using outside (a) and inside (b) primers. For each gene, the first lane is 1 kbp ladder DNA, the second is the gene product amplified by PCR in the wild-type MG1655 and the third lane is the same gene product amplified by PCR in the mutant CRT028. The smaller size of the gene product amplified using the outside primers and the absent gene product amplified using inside primer in the mutant confirmed the partial deletion of the genes Results are shown for ldhA, frdA, poxB, pta, adhE, pykF, zwf, and maeB.

FIG. 8. Growth characteristic of mutant CRT028 and mutant CRT028 expressing plasmid pACMNOx (a) Cell growth (OD₆₀₀ₙₘ) vs. time. (b) Specific growth rates. The experiments were conducted in baffled shake flasks containing minimal medium supplied with glucose under aerobic conditions. Each value represents the mean of the results of duplicate experiments.

FIG. 9. Yield of diapolycopendial and diapolycopendioic acid of wild-type MG1655/pACMNOx and mutant CRT028/pACMNOx in aerobic batch shake flask (a), carotenoid production profile of wild-type MG1655/pACMNOx () and mutant CRT028/pACMNOx () in aerobic batch bioreactor, cell culture picture of wild-type MG1655/pACMNOx and mutant CRT028/pACMNOx is shown in the upper left corner (b), production of carotenoid vs. consumed glucose of wild-type MG1655/pACMNOx () and mutant CRT028/pACMNOx () in batch bioreactor experiment (c) and time profiles of biomass production of wild-type MG1655/pACMNOx () and mutant CRT028/pACMNOx () in batch bioreactor (d). Overall carotenoid yield was presented as the slope of the regression line. Yield on glucose of wild-type and mutant are 0.04±0.00 and 0.17±0.04 mg-carotenoids/g-glucose respectively. The results are based on an average of three collecting samples.

FIG. 10. Production of diapolycopene (▪), tetradehydrolycopene (▪), tetradehydrolycopendial (▪) and lycopene (▪) by wild-type MG1655 (empty bar) and mutant CRT028 (filled bar) in aerobic batch shake flask. Product yield was reported in mg-carotenoid/g-glucose. Carotenoid content was estimated using extinction coefficient and appropriate dilution factor. Extinction coefficient of diapolycopene at a wavelength of 470 nm was obtained from Wieland et al, 1994. Extinction coefficient of tetradehydrolycopene at a wavelength of 508 nm and tetradehydrolycopendial at a wavelength of 504 nm were estimated from extinction coefficient values of diapolycopene and diapolycopendial respectively by adjusting based on number of carbons since they have similar structure. Extinction coefficient of lycopene at a wavelength of 475 nm was estimated from Britton 1995.

### DETAILED DESCRIPTION

E. coli are described herein that are highly efficient at producing carotenoids. In some embodiments, the E. coli comprises exogenous nucleic acids for expressing a carotenoid that the E. coli requires as necessary condition for the E. coli to reproduce. Further, the E coli may be made to be free of metabolic pathways that receive glucose as an input and do not result in production of the carotenoid. Other embodiments are directed to E. coli comprise exogenous nucleic acids for expressing a carotenoid and lacks, in any combination, one or more of the genes ldhA, frdA, poxB, pta, adhE, pykf, zwf, and maeB.

In one embodiment, diminishment or abrogation of one expression of one or more of the genes ldhA, frdA, poxB, pta, adhe, pykf, zwf, and maeB gene results in enhanced carotenoid production of between about 5 and about 500% or more over the production of the parental strain; artisans will immediately appreciate that all the ranges and values within the explicitly stated ranges are contemplated, e.g., at least about 100%, at least about 200%, at least about 300%, from about 100% to about 450%; or at least about 10% to at least about 200%. The parental strain refers to the E. coli that is modified to receive the inhibition and/or abrogation; as is evident, parental E. coli used to produce carotenoids may be improved. In another embodiment, diminishment or abrogation of the indicated gene results in E. coli results in an enhanced carotenoid production that is between about 5 and about 500% or more than wild-type production; artisans will immediately appreciate that all the ranges and values within the explicitly stated ranges are contemplated, e.g., at least about 100%, at least about 200%, at least about 300%, from about 100% to about 450%; or at least about 10% to at least about 200%. In some embodiments, CRT028/pACMNOx may be used as a benchmark for the wild-type.

One embodiment is an E. coli that provides a yield of at least about 0.1 or at least about 0.15 mg-carotenoid/g-glucose. The E. coli are made with the diminished or abrogated genes and cultured, followed by separation of the carotenoid.

One embodiment is an engineered E. coli for carotenoid production comprising exogenous nucleic acids for expressing a carotenoid that the E. coli requires as necessary condition for the E. coli to reproduce. Another embodiment is an E. coli that is free of metabolic pathways that: (i) receive glucose as an input and (ii) do not result in production of the carotenoid.

The term gene refers to a nucleic acid fragment that is capable of being expressed as a specific protein, including regulatory sequences preceding (5′ non-coding sequences) and following (3′ non-coding sequences) the coding sequence. The term native gene refers to a gene as found in nature with its own regulatory sequences. The term chimeric gene refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. The term endogenous gene refers to a native gene in its natural location in the genome of an organism. An exogenous gene refers to a gene not normally found in the host organism, but that is introduced into the host organism by gene transfer. Exogenous genes can comprise native genes inserted into a non-native organism, or chimeric genes. A transgene is a gene that has been introduced into the genome by a transformation procedure.

The genetically engineered E. coli may be engineered to be have diminished or abrogated expression of the indicated genes via any means as will be known to one skilled in the art. Abrogated gene expression refers to removing all expression of the gene. Diminished refers to reducing the quantity of expression, the activity, or the function of at least one polypeptide expressed by the gene. One embodiment is to delete the gene. Another embodiment is to delete a portion of the gene required for expression of a protein or proteins encoded by the genes. Another embodiment is to disrupt the gene to prevent its expression. Another embodiment is to suppress the expression of a protein or proteins encoded by the genes. Another embodiment is to include factors that suppress a product of the gene after it has been expressed. Persons or ordinary skill in these arts will be able to diminish or abrogate expression of the genes after reading this disclosure. US Pub No. 20060121558, WO 2004/101746 (PCT/US2004/014180), and WO 2006/091924 (PCT/US2006/006793) are hereby incorporated by reference herein, with the present specification controlling in case of conflict.

In one embodiment, diminishment of expression, activity, or function is effected via the use of antisense oligonucleotides, which are chimeric molecules, containing two or more chemically distinct regions, each made up of at least one nucleotide. These chimeric oligonucleotides contain, in one embodiment, at least one region wherein the oligonucleotide is modified so as to confer upon the oligonucleotide, in one embodiment, an increased resistance to nuclease degradation, or, in another embodiment, increased cellular uptake, and/or, in another embodiment, an increased binding affinity for the target polynucleotide. An additional region of the oligonucleotide may serve as a substrate for enzymes capable of cleaving RNA:DNA or RNA:RNA hybrids, which according to this aspect of the invention, serves as a means of gene silencing via degradation of specific sequences. Cleavage of the RNA target can be detected, in one embodiment by gel electrophoresis or, in another embodiment via nucleic acid hybridization techniques known in the art.

In one embodiment, inhibition as described may be effected via the use of a plasmid, which facilitates expression of the nucleic acid inhibitor. In one embodiment, the plasmid will comprise a regulatory sequence, which regulates expression of the nucleic acid inhibitor. In one embodiment, these regulatory sequences will comprise a promoter, which in one embodiment, provides for constitutive or, in another embodiment inducible, expression of the nucleic acid inhibitor. In another embodiment, the promoter may provide a means of high and low levels of expression of the nucleic acid inhibitor. In another embodiment, expression of the nucleic acid inhibitor may be regulated to provide for gene inactivation at a specific point in the growth stage of the cell.

Any E. coli which can produce carotenoids, and wherein the indicated genes may be diminished or abrogated to result in enhanced carotenoid production, is contemplated. Methods for modifying E. coli to produce carotenoids are known to those familiar with these arts. One method requires transfecting cells with exogenous nucleic acids that produce carotenoids. There are a number of techniques known in the art for introducing vectors into cells, such as, but not limited to: direct DNA uptake, virus, plasmid, linear DNA or liposome mediated transduction, receptor-mediated uptake and magnetoporation methods employing calcium-phosphate mediated and DEAE-dextran mediated methods of introduction, electroporation or liposome-mediated transfection.

In some embodiments, E. coli produces one or more carotenoids in the group consisting of diapolycopendial (DPL), diapolycopendioic acid (DPA), lycopene, tetradehydrolycopene, tetradehydrolycopendial, and/or any combinations thereof. Some E. coli were made to produce DPL or DPA with high efficiency.

There are over 600 known carotenoids; they are split into two classes, xanthophylls and carotenes. Further, microorganisms may be engineered to make non-naturally occurring carotenoids. A carotenoid refers to any carotenoid group, including. e.g., myxobacton, spheroidene, spheroidenone, lutein, violaxanthin, 4-ketorulene, myxoxanthrophyll, echinenione, canthaxanthin, phytoene, alpha-, beta.-, gamma-, delta.- or epsilon-carotene, lycopene, beta-cryptoxanthin monoglucoside or neoxanthin. In another embodiment, the carotenoids may include antheraxanthin, adonixanthin, astaxanthin, canthaxanthin, capsorubrin, .beta.-cryptoxanthin echinenone, zeta-carotene, alpha-cryptoxanthin, diatoxanthin, 7,8-didehydroastaxanthin, fucoxanthin, fucoxanthinol, isorenieratene, lactucaxanthin, lutein, lycopene, neoxanthin, neurosporene, hydroxyneurosporene, peridinin, phytoene, rhodopin, rhodopin glucoside, siphonaxanthin, spheroidene, spheroidenone, spirilloxanthin, uriolide, uriolide acetate, violaxanthin, zeaxanthin-.beta.-diglucoside, and zeaxanthin.

Typically, carotenoids are produced by providing a microorganism and culturing the provided microorganism with a suitable culture medium. In general, the culture; media and/or culture conditions can be such that the microorganisms grow to an adequate density and produce carotenoids efficiently. For large-scale production processes, any method can be used such as those described elsewhere (Manual of Industrial Microbiology and Biotechnology, 2n Edition, Editors: A. L. Demain and J. E. Davies, ASM Press; and Principles of Fermentation Technology, P. F. Stanbury and A. Whitaker, Pergamon). Briefly, a large tank (e.g., a 100 gallon, 200 gallon, 500 gallon, or more tank) containing appropriate culture medium with, for example, a glucose carbon source is inoculated with a particular microorganism. After inoculation, the microorganisms are incubated to allow biomass to be produced. Once a desired biomass is reached, the broth containing the microorganisms can be transferred to a second tank. This second tank can be any size. For example, the second tank can be larger, smaller, or the same size as the so first tank. Typically, the second tank is larger than the first such that additional culture; medium can be added to the broth from the first tank. In addition, the culture medium within this second tank can be the same as, or different from, that used in the first tank.

Once transferred, the microorganisms can be incubated to allow for the production of a carotenoid. Once produced, any method can be used to isolate the carotenoids. For example, common separation techniques can be used to remove the biomass from the broth, and common isolation procedures (e.g., extraction, distillation, and ion-exchange procedures) can be used to obtain the carotenoid from the biomass.

Previous attempts of improving carotenoid production in recombinant E. coli mostly relied on a non-systematic approach of genetic manipulation. This included over-expression of genes of the carotenoid biosynthesis pathway or deletion of competing central metabolic genes to redirect precursors towards enhanced production of carotenoids (2, 24, 10). For instance, US Pub No. 2006/0121558 discloses a series of genes to be overexpressed or deleted. However, this strategy is inefficient due to the lack of direction and an extensive amount of time and cost required. In addition, this approach may not capture all genetic modifications needed for an efficient carotenoid formation.

The identifications of the targets of gene manipulations resulting in a specific cellular metabolism to function in a favorable direction is challenging due to the complexity of the interconnected cell reaction network. In fact, in one E. coli mutant, the MG1655, over 29,000 pathways exist; processes to select or optimize these pathways have unpredictable outcomes.

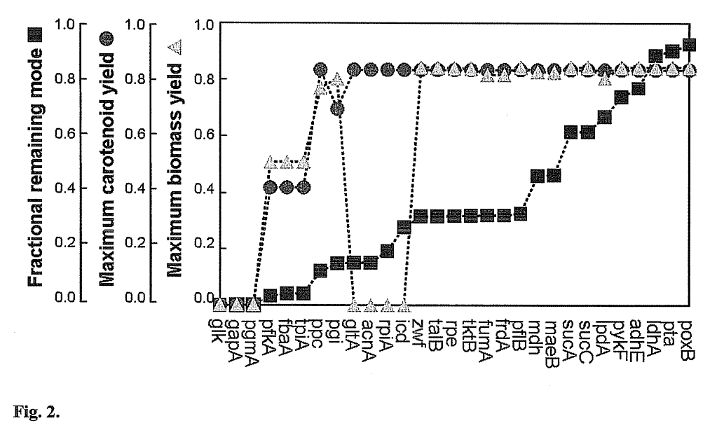

One embodiment herein is the use of EMA to rationally design an E. coli mutant strain for an enhanced production of a carotenoid. The carotenoid may be specifically chosen or a more general optimization may be performed. By way of example, the carotenoids diapolycopendial (DPL) and diapolycopendioic acid (DPA) were chosen and this process is described herein. An embodiment of a design process is to generate all possible pathways that can exist in a cell by EMA analysis. Gene knockouts which result in zero yield of biomass are considered lethal and tare not deleted from the cell. The target genes are identified when elimination of that gene still maintains the maximum possible yield (or a predetermined minimum yield, e.g., at least 50%, at least 70%, at least 80%. or at least 90%) of carotenoid product and retains a reasonable yield of biomass (e.g., at least 10%, at least 20%, at least 30%, at least 40%, at least 50%, at least 60%, or at least 70%) while the largest possible number of elementary modes is eliminated (minimum number of Factional remaining modes). As at FIG. 2, a seriatim of remaining modes may be made to evaluate the number of remaining modes.

The modes in which production of biomass and the carotenoids or selected carotenoids are coupled may be identified. In this manner, other pathways wherein there is no such linkage may be eliminated and only pathways with this linkage may be preserved, resulting in creation of an E. coli with linkage between carotenoid production and biomass production.

A specific example of these processes is provided herein. Elementary Mode Analysis (EMA) was performed on a recombinant metabolic network of carotenoid producing E. coli in order to identify multiple gene knockouts for an enhanced synthesis of carotenoids. The process was chosen to specifically choose diapolycopendial (DPL) and diapolycopendioic acid (DPA). In this example, all inefficient carotenoid biosynthesis pathways were eliminated in a strain by way of making a combination of eight gene deletions. To validate the model prediction, the designed strain was constructed and tested for its performance. The designed mutant produced the carotenoids at significantly increased yields and rates as compared to the wild-type. The consistency between model prediction and experimental results demonstrated that Elementary Mode Analysis was effective. The rationally designed mutant was experimentally constructed and the kinetics of the production of DPL and DPA was determined to verify the model predictions.

The Elementary Mode Analysis (EMA) used nullspace and convex analysis under steady state balance operation of the network to decompose the complex metabolic network of a cell into a set of unique and indivisible pathways, called elementary modes (15, 17). As a result, the EMA generated all possible pathways that can exist in a cell. This pathway information along with the direct correspondence between metabolic reaction network and genetic network allowed the prediction of the cellular phenotype resulting from genotype perturbations. EMA has been previously used for understanding the yield range of metabolic pathways in a network or for predicting cell phenotype in a genetically modified organism. For example, EMA has been applied for examining the effects of gene manipulations and of changes in environmental conditions on E. coli cell growth (19). EMA has also been used in metabolic engineering. The method can be used for designing the intermediary metabolism of a cell for cell growth and biomass production (8, 9, 21) or to for the formation of primary metabolites such as ethanol (22). However, previous examples of EMA applications do not include applications to recombinant cells containing a complex pathway for a secondary metabolite.

### Materials and Methods

Bacterial strains and plasmids. E. coli K12 MG1655 and its mutant derivatives were used as the hosts for synthesis of carotenoids DPL and DPA. Individual strains containing respectively the genetic knockouts ΔldhA::kan+, ΔfrdA::kan+, ΔpoxB::kan+, Δpta::kan+, ΔadhE::kan+, ΔpykF::kan+, Δzwf::kan+ and ΔmaeB::kan+ were obtained from the E. coli knockout library, the Keio collection (6). E. coli cells containing multiple-gene knockouts generated in this study were constructed using a generalized PI transduction technique after transferring the desired deletion gene from the knockout library into the MG1655 recipient strain (5). Table 1 summarized all strains used in this study. Primers designed to regions outside and inside of the targeted knockout gene shown in Table 2 were used in conjunction with PCR and gel electrophoresis to verify the knockout construction. The inside primers verify that the structural gene is absent in the chromosome while the outside primers validate that the targeted gene is indeed deleted. The strains were later transformed with a carotenoid plasmid, pACMNOx (14). Included on the plasmid is the chloramphenicol resistance gene for selection.

Growth Medium. The bacterial cells were cultivated in M9 minimal medium (2.0 g/l NH₄Cl, 1 g/l NaCl, 25.6 g/l Na₂HPO₄.7H₂O, 6.0 g/l KH₂PO₄, 0.01 g/l CaCl₂, 0.24 g/l MgSO₄ (Sigma, St. Louis, Mo.)) supplemented with trace metals and minerals (5.5 mg/l CaCl₂.2H₂O, 1.0 mg/l MnCl₂.4H₂O, 1.7 mg/l ZnCl₂, 0.43 mg/l CuCl₂, 0.60 mg/l CoCl₂.6H₂O, 0.60 mg/l NaMoO₄.2H₂O, 8.42 mg/l FeSO₄.7H₂O, 10 mg/l Fe(NH₄)-citrate, 0.38 mg/l Na₂B₄O₇.10H₂O, 1 mg/l of thiamine (Sigma, St. Louis, Mo.)). The medium contained 5 g/l of glucose as a carbon source. The addition of 50 μg/ml chloramphenicol was used to maintain plasmid in the carotenoid-producing cell culture.

Growth in batch shake-flasks and bioreactors. In shake-flask growth experiment, 1 ml of seed culture grown overnight from transformed colonies was inoculated in a 250 ml baffled shake flask containing 50 ml of M9 medium supplemented with glucose. The culture was aerobically grown in a Labline shaking incubator at 200 rpm agitation speed. Temperature was maintained at 30° C. Cell samples (5 ml) were periodically collected for carotenoid measurement. Batch bioreactor experiments were conducted in a 101 Braun bioreactor (Biostat M D, B. Braun Biotech International, Melsungen, Germany) containing 51 of the same culture medium as the shake flask experiments. The culture was inoculated with 1% (v/v) seed culture grown in aerobic baffled shake flasks. The reactor was operated aerobically at 1 vvm aeration rate. Agitation speed was set at 300 rpm and temperature was controlled at 30° C. pH was controlled at 6.9 with 28% (v/v) NH₄OH and 40% (v/v) H₃PO₄ (Sigma, St. Louis, Mo.). An online off-gas mass spectrometer (PRIMAδ-B MS; Thermo Onix, Houston, Tex.) was used for determining oxygen consumption and carbon dioxide formation in the culture. Cell samples and supernatants were collected every 2-4 hours for glucose, carotenoid and cell growth measurements.

Analytical techniques. Cell growth was monitored via optical density at a wavelength of 600 nm in a 1 cm cuvette using a Hewlett Packard 8452A Diode Array spectrophotometer (Palo Alto, Calif.). Cell mass was determined from a standard curve correlating optical density to cell dry weight. To measure cell dry weight, samples were collected from the culture, washed with cold deionized water after centrifugation and transferred to a pre-weighed tube. The tube containing cell pellet was dried at 100° C. for 24 hours, then weighed and the mass of the empty tube was subtracted to determine cell dry weight concentration in the culture. Metabolite concentrations including glucose and other secreted byproducts were determined using a HPLC system (SHIMADZ10A, Shimadzu, Columbia, Md.) equipped with an autosampler (SIL-10AF), a cation exchange column (HPX-87H, Biorad Labs, Hercules, Calif.) and two detectors in series including a UV-vis detector (SPD-10A) and a refractive index detector (RID-10A). The column was run in an isocratic mode at 50° C. at a flow rate of 0.6 ml/min with 0.01 M H₂SO₄ as a mobile phase. A standard curve correlating area to concentration of metabolites was used to determine the quantity of metabolites in the sample.

Carotenoid analysis. Intracellular carotenoids were extracted from 5 ml cell culture. The centrifuged cell pellet was first washed with 1 ml of cold 0.9% NaCl solution. The washed cell pellet was then extracted with 1 ml acetone with vigorous vortexing for 5 minutes. After centrifugation, the remaining cell pellet was resuspended in 10% (g/g) KOH and incubated at 30° C. for 24 hours. The KOH extract solution was later purified by liquid-liquid extraction with chloroform. Carotenoid content in acetone and KOH extract solution was quantified by measuring the absorbance at 506 nm and 490 nm respectively. Concentration of carotenoid was determined using extinction coefficients obtained from CaroteNature (Lupsingen, Switzerland) and appropriate dilution factor. The extinction coefficient of diapolycopendial at 506 nm and diapolycopendioic acid at 490 nm are 4084 and 2784 (g/100 ml)⁻¹ cm⁻¹ respectively.

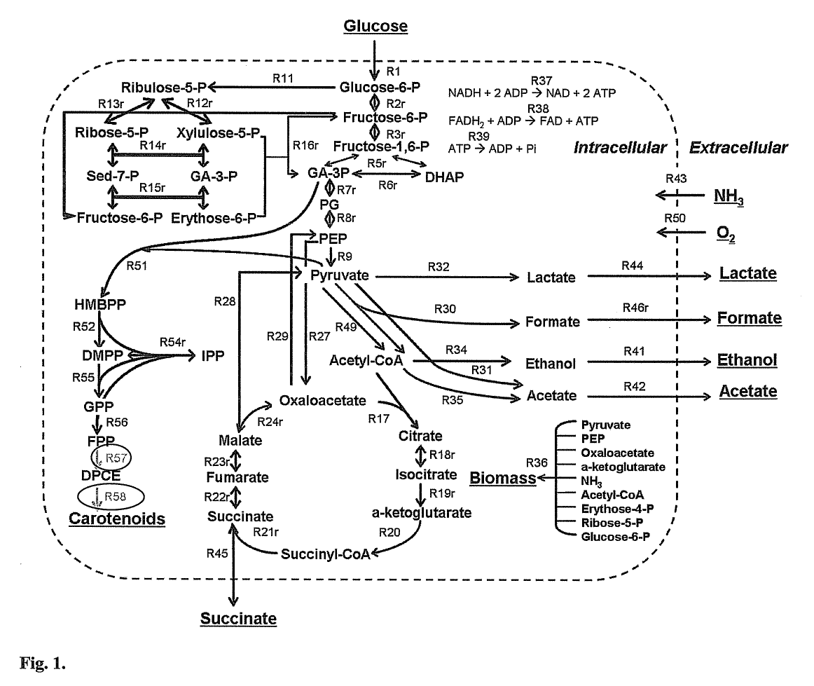

Elementary Mode Analysis. The metabolic network was based on the previously described E. coli network (Carlson et al 2004a). Carotenoid synthesis through the non-mevalonate pathway was included in the model to account for the production of carotenoid in the recombinant cells. The pathway details were collected from the Ecocyc database that is available at www.ecocyc.org. The model was based on utilizing glucose as the carbon source. Cell growth in the metabolic network is described through the production of biomass which is formed from precursors and energy draining of the central metabolic pathway of E. coli (11). FIG. 1 shows the metabolic map of the carotenoid producing E. coli used in this study. The model has been analyzed using METATOOL software version 3.9.2 (15). Elementary mode results were analyzed using Excel Microsoft Corp. for mode sorting and filtering.

### Results

Identification of target gene knockouts. A carotenoid expressing E. coli metabolic model was first constructed by adding the biosynthesis pathway of carotenoids DPL and DPA into the central metabolic network of E. coli MG1655 which was used as the host for synthesizing carotenoids. FIG. 1 presents the metabolic network of carotenoid expressing E. coli considered. It included a total of 58 metabolic reactions (22 reversible, 36 irreversible) and 57 metabolites. Elementary Mode Analysis on the network revealed a total of 29,532 elementary modes. Each mode represents a unique, possible pathway with balanced metabolites and cofactors. A total of 24,155 modes is aerobic, while 5,377 modes exist under anaerobic conditions. There are 5,923 total modes in which production of biomass and carotenoids diapolycopendial and diapolycopendioic acid are coupled. The large number of elementary modes illustrates the flexibility and robustness of the cell to adapt itself to particular conditions by using pathways that provides the optimal fitness. Comparison of all elementary modes revealed a maximum carotenoid yield of 0.83 carbon mole of carotenoid per carbon mole of glucose. The maximum possible biomass yield was 0.84 carbon mole of biomass per carbon mole of glucose, consistent with previous results (Carlson et al 2004).

An evaluation of gene knockout effects on cell phenotype was performed. Gene knockouts were simulated by removing the enzymatic reaction corresponding to that gene from the stoichiometric matrix. The phenotype of that specific knockout mutant was then represented by a combination of remaining elementary modes when that reaction was deleted. Specifically, gene knockout effects were chosen based on: cell viability (biomass yield), and maximal yield of carotenoid and fraction of remaining modes after each individual single gene knockout in which a specific gene and its corresponding reaction was eliminated from the metabolic network. The effects of the single gene deletions are summarized in FIG. 2. All gene knockouts were sorted in increasing orders of the fraction of remaining elementary modes. Gene knockouts which result in zero yield of biomass are considered lethal and therefore, these were not deleted from the cell. The result in FIG. 2 assists the process of screening and selecting potential gene knockout targets. Among the set of all individual knockouts, the target genes are identified when elimination of that gene still maintains the maximum possible yield of carotenoid product and retains a reasonable yield of biomass while the largest possible number of elementary modes is eliminated (minimum number of fractional remaining modes). For example, zwf is a target for deletion since deletion of this gene eliminates more than 60% of identified elementary modes and still supports high yield of carotenoid and biomass. This approach allows the determination of optimal gene knockout targets which eliminate the largest number of elementary modes without affecting the most efficient pathway for carotenoid and biomass synthesis. This identification tool was applied in a sequential manner for selecting an optimal combination of multiple gene knockouts. That is, the identified gene knockout from previous steps was used as the genetic background in the next steps for determining additional gene knockouts. This process was continued until no further elementary modes can be eliminated without reducing the maximum yield of carotenoid production. The combination of these gene knockout targets, therefore, forces the cell to function according to the remaining efficient carotenoid-producing elementary modes. Table 3 summarizes the number of remaining elementary modes when each gene knockout target was sequentially combined.

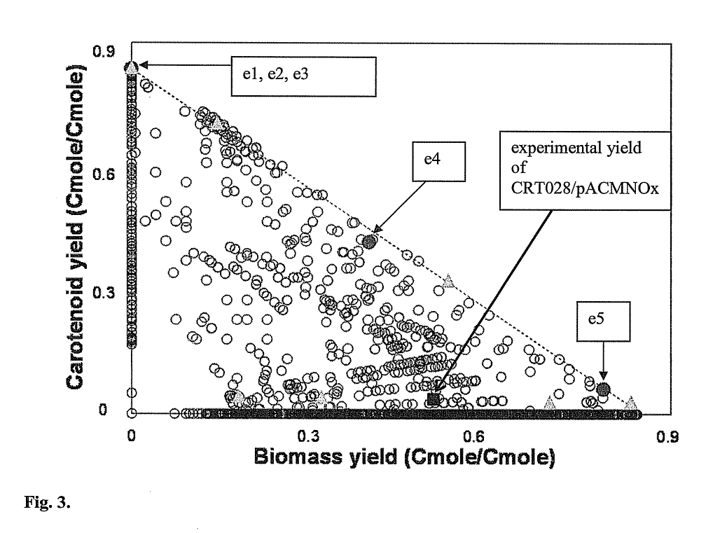

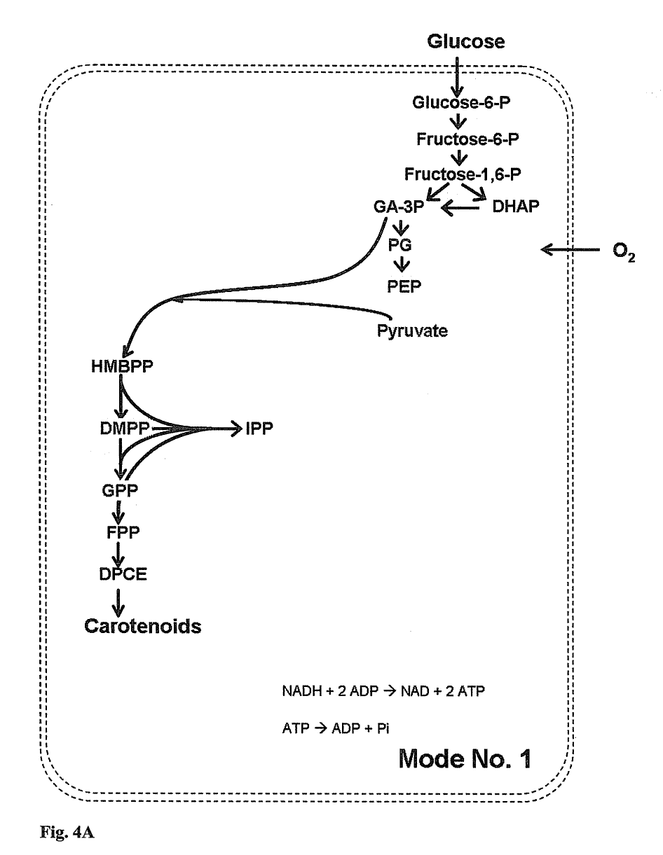

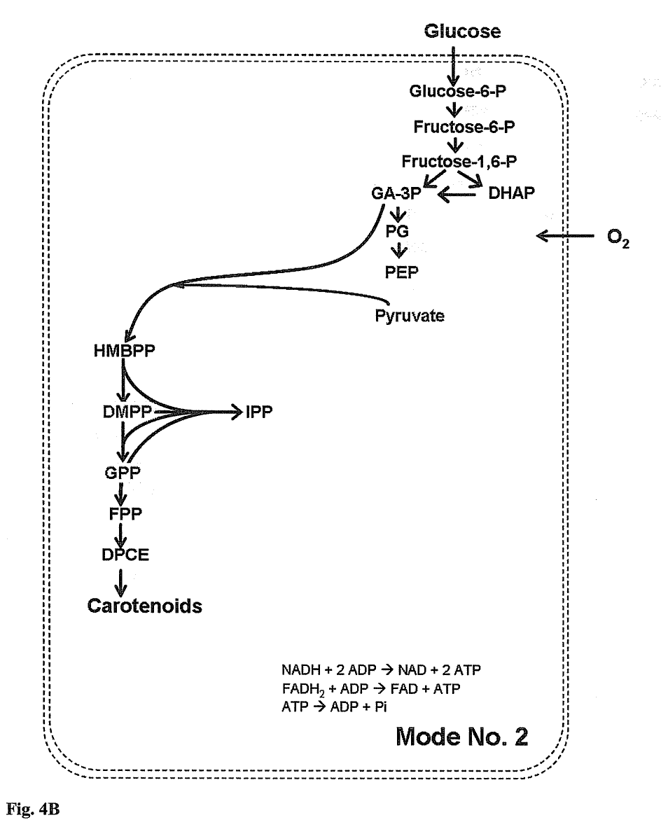

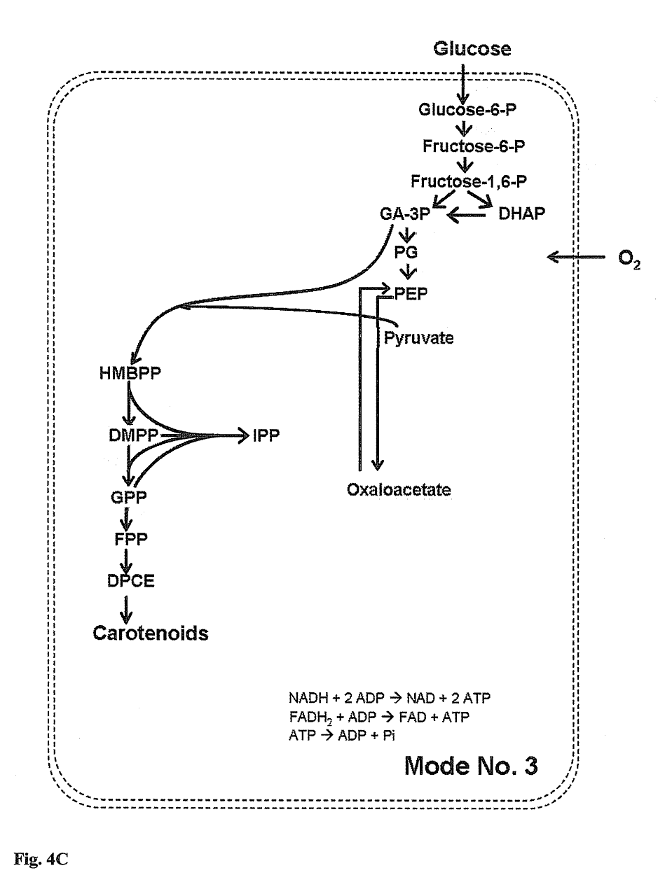

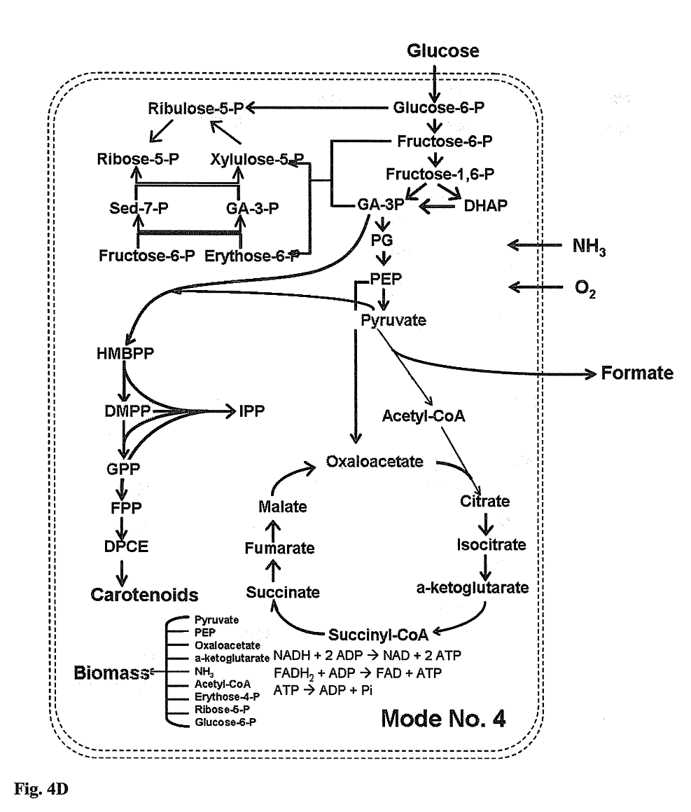

The identification approach resulted in a combination of eight gene knockouts which predicted a likely over-production of the carotenoids DPL and DPA in E. coli. The gene deletions included the removal of byproduct synthesis genes for lactate, succinate, acetate and ethanol which were experimentally accomplished by disrupting lactate dehydrogenase (ldhA; reaction R32), fumarate reductase (frdA; reaction R22), pyruvate oxidase poxB; reaction R31), phosphate acetyltransferase (pta; reaction R35) and alcohol dehydrogenase (adhE; reaction R34) respectively. Other target genes were pyruvate kinase (pykf; reaction R9), glucose-6-phosphate-1-dehydrogenase (zwf; reaction R11) and malate dehydrogenase (maeB; reaction R28). The reaction designation of these eight knockout targets is summarized in Table 4. The combination of these gene knockouts reduces the number of elementary modes to five remaining modes as shown in FIG. 3. The overall reaction stoichiometry and detailed pathways of these elementary modes are shown in Table 5 and FIG. 4 respectively. All of these remaining modes include carotenoid synthesis. Therefore, the knockout mutant is expected to produce the carotenoids DPL/DPA more efficiently than the wild-type which contains many modes that do not produce carotenoids at all. In two of the remaining modes, carotenoid synthesis is coupled with biomass formation.

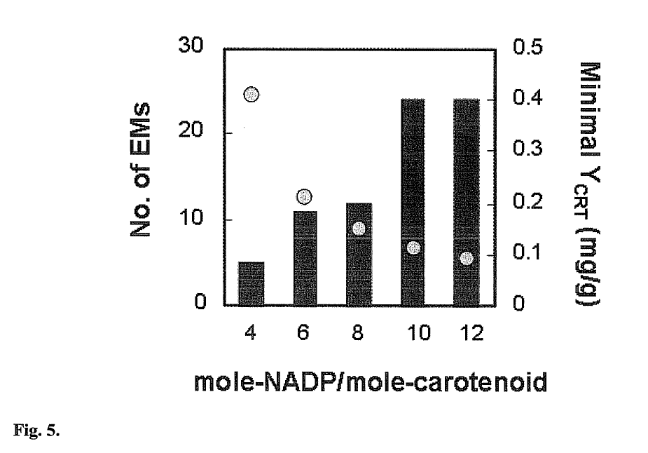

Effect of NADP on production of diapolycopendioic acid. Since a detailed biosynthesis pathway of diapolycopendioic acid (DPA) has not yet been established, it is unclear how many NADP cofactors are required for the synthesis of one mole of DPA. Therefore, the effect of the number of NADP cofactors on the number of available modes and predicted yields of DPA in the host background of CRT028/pACMNOx (FIG. 5) was evaluated with the assumption that 4 NADP are required per mole of DPA formed. This number was selected based on the assumption that for every atom of hydrogen lost in the oxygenation step, one mole of NADP cofactor is required. EMA showed that the change of the number of NADP has no effect on the predicted maximum yield of DPA. However, an increased number of NADP cofactors required for synthesis of one mole of DPA results in an increased number of available elementary modes and a decreased minimum predicted yield of DPA. Surprisingly, all elementary modes available in CRT028/pACMNOx are all directed to the production of DPA regardless of the number of NADP chosen. This confirmed that for all numbers of NADP assumed, CRT028/pACMNOx is expected to produce DPA.

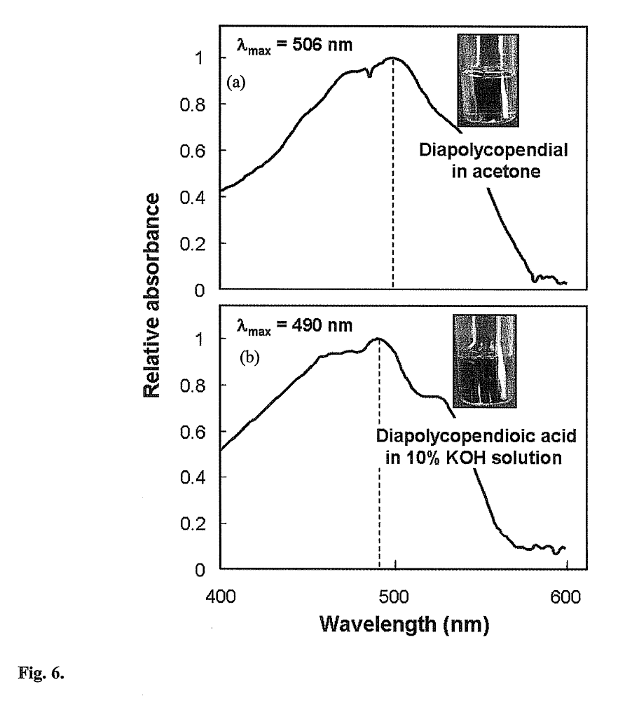

Quantification of diapolycopendial and diapolycopendioic acid. Three carotenogenic genes crtM, crtN and crtOx were responsible for the synthesis of the end product, diapolycopendioic acid. A previous study has shown that E. coli expressing plasmid pACMNOx containing these genes stored the end product as well as its intermediates, diapolycopendial in the cell (13). These two products have distinct solubilities. Diapolycopendial is soluble in acetone while diapolycopendioic acid is soluble in an aqueous KOH solution. As a result, two extraction processes were developed to extract each of them from the cell membrane. Diapolycopendial was first extracted from the cells using acetone. A 10% KOH solution was then used for the second extraction process to isolate the carotenoid diapolycopendioic acid. Absorption spectra of acetone and KOH extracts using a spectrophotometer are shown in FIG. 6. The distinct absorption spectra of the extract solutions confirmed that different carotenoids were being dissolved in each solution. In acetone supernatant, the maximum absorbance was found at a wavelength 506 nm, while maximum absorbance in KOH supernatant was at a wavelength of 490 nm. These observed wavelengths agree with the wavelength reported by in Tao et al. (2005), confirming the presence of carotenoid, diapolycopendial in acetone extract (reported λmax, 507 nm) and diapolycopendioic acid in KOH extract (reported λmax, 490 nm). Since both carotenoids, DPL and DPA, were found in the cells, measured carotenoid product are given as the sum of both carotenoids.

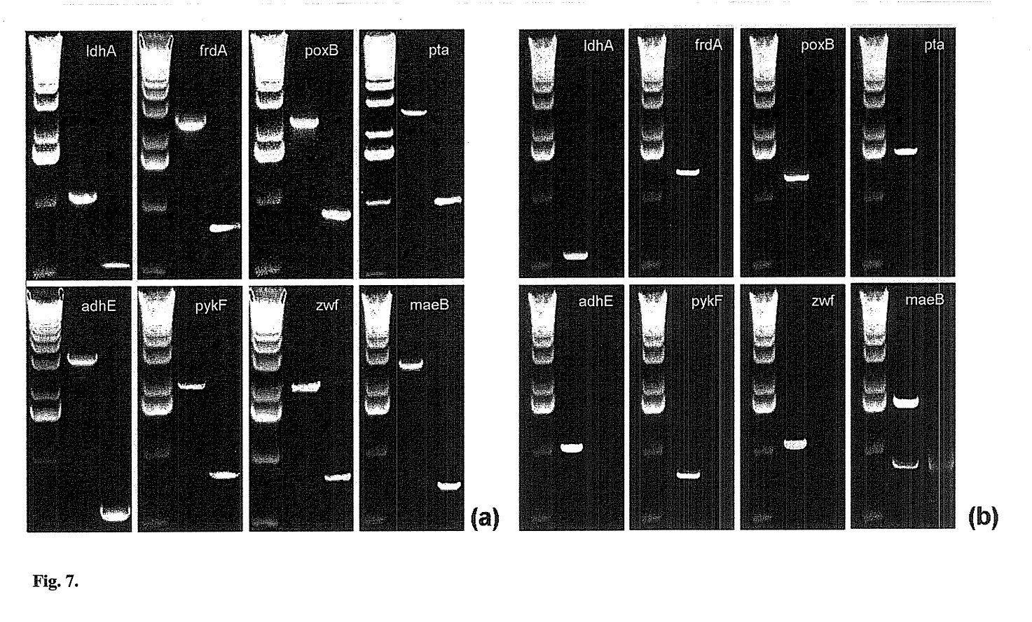

Strain construction. To validate the knockout strain design based on EMA, the designed mutant strain CRT028, was constructed using established methods (5) and verified by PCR amplification and gel electrophoresis using both inside and outside primers specific to that deleted genes. Inside primers specified to the deleted part of the gene were used to ensure that a disrupted gene was not accidentally displaced somewhere else on the chromosome. Outside primers specified to undeleted portions of the gene were used to verify gene disruption at the known locations on the E. coli chromosome. The gel pictures in FIG. 7 confirmed of the gene disruptions in the mutant CRT028 as compared with the wild-type MG1655 containing undisrupted genes. Using the outside primers, the smaller size PCR product of the mutant as compared with that of the wild-type confirmed the gene disruption. In addition, the absence of gene product amplified using inside primers in the mutant confirmed the complete deletion of eight genes from its chromosome.

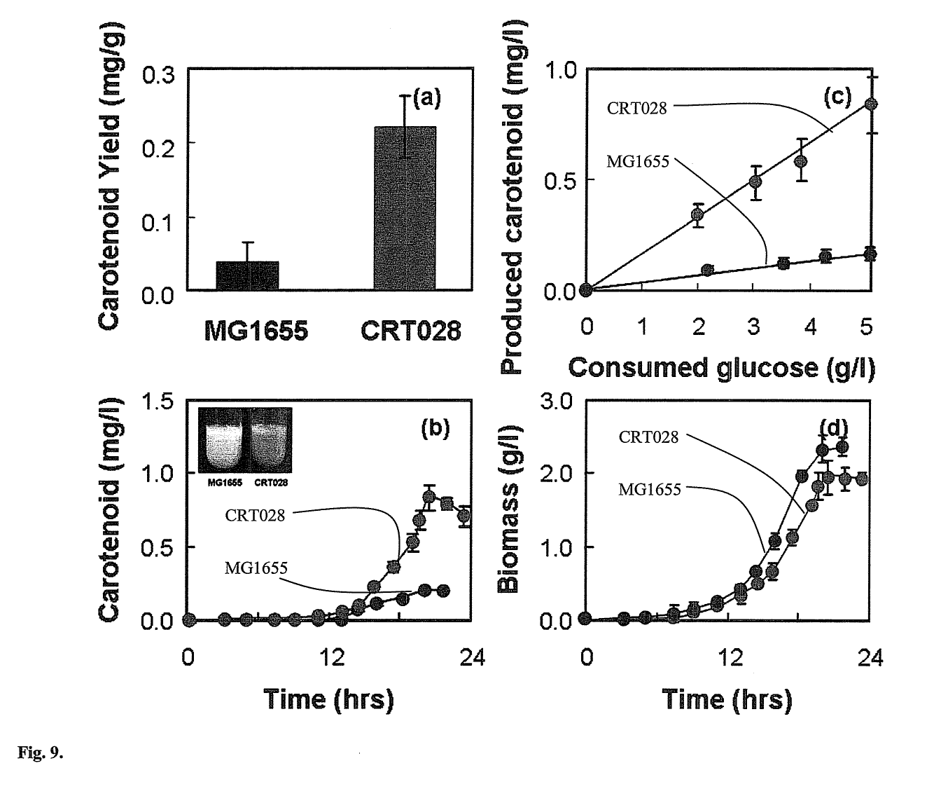

Strain characterization. According to the strain design by EMA, two of the remaining modes in the mutant are growth-associated carotenoid producing modes (Table 5) which suggests a tight coupling between carotenoid synthesis and cell growth in the mutant. Therefore, whether or not cell growth and carotenoid production are indeed coupled in the mutant strain was tested. Growth experiments in aerobic shake flasks showed that the mutant CRT028 grew very slowly without the plasmid pACMNOx and growth was restored when the plasmid was introduced into the mutant (FIG. 7). The growth rate of the mutant with plasmid was approximately 4-fold faster than that of the mutant without plasmid. Thus, carotenoid formation provided a strong selection pressure for plasmid even in the absence of antibiotics. Carotenoid yield (mg of carotenoid produced per g of glucose consumed) of both E. coli wild-type MG1655/pACMNOx and mutant strain CRT028/pACMNOx were measured. The experiments were conducted in both shake-flasks and in batch bioreactors. FIG. 9a presents the yield of DPL and DPA in wild-type and in the constructed knockout strain determined in aerobic shake flask experiments. Carotenoids production in the mutant CRT028/pACMNOx is significantly higher than in the wild-type MG1655/pACMNOx. In aerobic batch bioreactors, the mutant yielded 0.17±0.04 mg-carotenoid/g-glucose compared to 0.04±0.00 mg-carotenoid/g-glucose by the wild-type (FIG. 9c). The production profile of total DPL and DPA in batch bioreactors shown in FIG. 9b also revealed that the mutant produced carotenoid at faster production rate than the wild-type. In addition, comparison of the growth curves of MG1655/pACMNOx and CRT028/pACMNOx suggested that the mutant grew slower than the wild-type with the specific cell growth rate reduced by 23%. Growth and carotenoid phenotype of both wild-type and mutant are summarized in Table 6. The results from the online off-gas mass spectrometer also revealed in the case of the mutant a decrease in carbon dioxide production rate but a significantly increased oxygen uptake rate as compared to the wild-type.

Strain comparison. Several E. coli strains have been previously developed to improve production of carotenoid through multiple gene modifications. To compare the differences between the improved strain CRT028/pACMNOx and other mutant E. coli strains developed previously, EMA was applied to evaluate the effects of the gene knockouts on the production of DPA and on the reduction of inefficient pathways for carotenoid production (Table 7). The results show that the mutants containing gene knockouts previously identified for enhanced production of carotenoid still contain a significant number of inefficient carotenoid-producing pathways. These remaining inefficient pathways likely reduce the yield of carotneoid if the pathways are used. Unlike each of these mutants, CRT028/pACMNOx contains only carotenoid-producing elementary modes. Therefore, our designed strain is expected to be able to efficiently produce carotenoid diapolycopendioic acid.

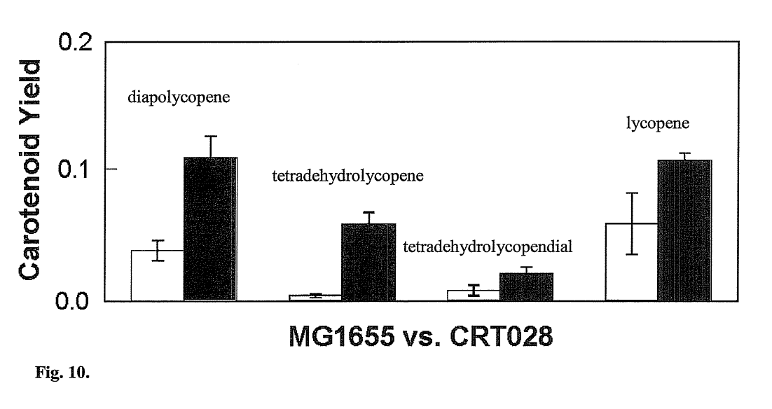

Production of other carotenoids. Since all carotenoids are known to share a common biosynthesis pathway, the performance of the improved mutant was also tested for the production of other carotenoids including diapolycopene, tetradehydrolycopene, tetradehydrolycopendial and lycopene. The comparison of each carotenoid yield (mg-carotenoid/g-glucose) between wild-type and the mutant shown in FIG. 10 demonstrates that the mutant outperforms the wild-type in the synthesis of other carotenoids as well. The yield improvement of all carotenoids tested in the mutant indicates that the improved mutant could generally be used as a platform host for the production of carotenoids, i.e., besides DPL and DPA.

Because carotenoid products are of considerable industrial and nutritional value, others have recently metabolically engineered recombinant cells for enhancing production of carotenoids. The strategies applied were based on overexpression and/or deletion of genes involved in the carotenoid biosynthesis pathway. Although improvement in carotenoid production was observed, the genetic manipulations in general did not offer the features designed into the improved mutant. Herein a method has been presented using Elementary Mode Analysis (EMA) to design an optimized E. coli strain for the most efficient synthesis of carotenoids. In particular, EMA was applied to identify targets for gene deletions resulting in efficient production of diapolycopendial (DPL) and diapolycopendioic acid (DPA). The designed strain was obtained through a sequential implementation of eight multiple gene deletions which narrowed the possible pathway space to the efficient carotenoid producing options.

Based on this analysis, an inverse relationship between the fermentation product formation and carotenoid production due to shared precursors between these products was created and observed. Without being bound to a particular theory of operation, removal of the identified fermentation product synthesis genes-ldhA, frdA, poxB, pta, adhE-directed more flux into the carotenoid synthesis pathway. Surprisingly, the pyruvate kinase; pykf was also an effective deletion. The pykf gene encodes for one of the two isoenzymes of pyruvate kinase which permits the inter-conversion between pyruvate and phosphoenolpyruvate. Deletion of pykF could have blocked the further metabolism of the carotenoid precursors, pyruvate and glycerol-3-phosphate leading to an increased glycolytic supply for the carotenoid synthesis pathway. The disruption of glucose 6-phosphate-1-dehydrogenase encoded by zwf seems to have diverted the carbon flux into the pool of carotenoid precursors and reduced carbon loss in form of carbon dioxide usually mediated by the pentose phosphate pathway (18). Deletion of malic enzymes maeB as suggested by EMA apparently prevented carotenoid precursors from draining into the anaplerotic pathway.

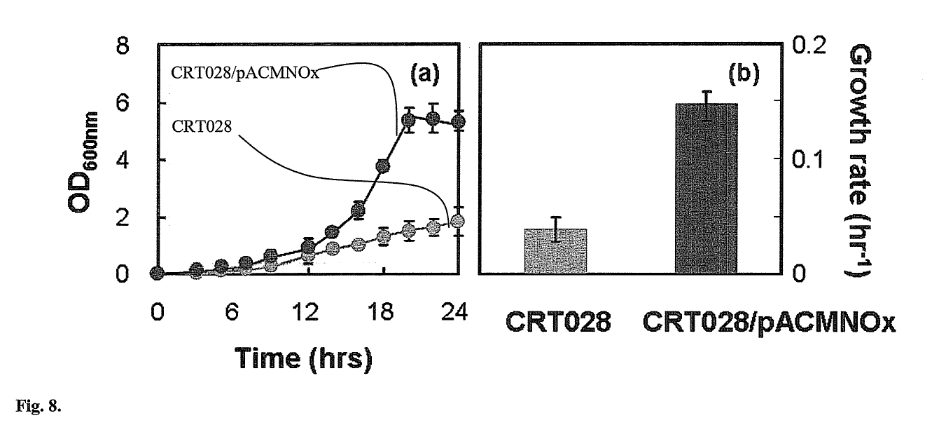

Experimental results confirmed that the synthesis of carotenoid is essential for cell growth in the mutant CRT028 (FIG. 8). The coupling between carotenoid synthesis and cell growth can be used as a natural selection pressure for maintaining carotenoid synthesis plasmids in the cells. In the designed mutant, the production of diapolycopendial (DPL) and diapolycopendioic acid (DPA) was improved as compared with the wild-type under identical growth condition. However, in the mutant, the specific growth rate is reduced. This is consistent with previous studies which showed a higher expression of carotenoid products in E. coli at reduced growth rates (4). The inverse relationship between production of carotenoid and biomass is also predicted by EMA. Moreover, the mutant requires a significantly higher amount of oxygen in comparison to the wild-type. The role of oxygen on carotenoid synthesis was also observed in the production of lycopene by Alper et al. (2006).

Testing the strain performance on the production of other carotenoids showed that the mutant CRT028 is better than the wild-type MG1655. The mutant was specifically optimized for the production of DPL/DPA and it performed at its best on the production of carotenoids DPL/DPA.

Based on the EMA prediction, mutant CRT028/pACMNOx can only function using a combination of the five remaining elementary modes with a theoretical minimum carotenoid yield of 0.41 mg-carotenoid/g-glucose. However, experimental yield observed in the mutant was lower than the predicted value. It is likely that the inconsistency between observed yield and predicted possible yield may be a result of an incorrect number of NADP cofactor chosen. As shown in FIG. 5, the predicted yield of DPA and numbers of available elementary modes depend on number of NADP cofactor assumed for synthesis of one mole of DPA. If eight moles of NADP required per mole of DPA was chosen in the model instead, the predicted minimum yield of DPA in the mutant CRT028/pACMNOx would be 0.15 mg-carotenoid/g-glucose which is consistent with the experimental yield observed.

In addition, there were other factors that could possibly prevent the mutant from reaching a higher yield of carotenoid products. Based on overall reaction stoichiometry of elementary modes remaining in the mutant (Table 5), high oxygen levels are required for product synthesis. It is likely that the supplied oxygen becomes limiting especially once the cell reaches a high cell density. Moreover, rate limiting steps in the downstream pathway of carotenoid biosynthesis could be responsible for preventing the cell from reaching higher product yields. There were several studies showing that overexpression of these downstream genes could lead to an increased yield. For example, production of carotenoids such as lycopene, torulene or beta-carotene were found to be improved when the isoprenoid biosynthesis genes such as dxs or idi were overexpressed in E. coli (13). The product formation could also be inhibited by some unknown negative effects of the cell regulatory system. Future studies will be needed for investigating these factors to further improve the product yield and productivities. The methods disclosed herein may be used to enhance carotenoid secondary metabolite production.

The following references are hereby incorporated by reference herein; in the case of conflict, the present specification controls.
1. Albrecht, M., S. Takaichi, N. Misawa, G. Schnurr, P. Boger, G. Sandmann. 1997. Synthesis of atypical cyclic and acyclic hydroxyl carotenoids in Escherichia coli transformants. J. Biotechnol 8:177-85
2. Albrecht, M., N. Misawa, G. Sandmann. 1999. Metabolic engineering of the terpenoid biosynthetic pathway of Escherichia coli for production of the carotenoids β-carotene and zeaxanthin. Biotechnology Letters 21: 791-95
3. Alper, H., J. Yong-Su, J. F. Moxley, G. Stephanopoulos. 2005. Identifying gene targets for the metabolic engineering of lycopene biosynthesis in Escherichia coli. Metab Eng. 7:155-64.
4. Alper, H., K. Miyaoku, G. Stephanopoulos. 2006. Characterization of lycopene-overproduing E. coli strains in high cell density fermentations. Appl Microbiol Biotechnol. 72: 968-74
5. Ausubel, F., R. Brent, R. E. Kingston, D. D. Moore, J. G. Seidman, J. A. Smith, K. Struhl. 1995. Short Protocols in Molecular Biology. New York. Wiley & Sons, Inc.
6. Baba, T., T. Ara, M. Hasegawa, Y. Takai, Y. Okumura, M. Baba, K. A. Datsenko, M. Tomita, B. L. Wanner, H. Mori. 2006. Construction of Escherichia coli K-12 in-frame, single-gene knockout mutants: the Keio collection. Mol Syst Biol. 2: 2006-0008.
7. Britton, G., S. Liaaen-Jensen, H. Pfander. Carotenoids. Spectroscopy, Birkauser Verlag Basel 1995. pp. 57-61.
8. Carlson, R., F. Srienc. 2004a. Fundamental Escherichia coli biochemical pathways for biomass and energy production: identification of reactions. Biotechnology & Bioengineering 85(1):1-19.
9. Carlson, R., F. Srienc. 2004b. Fundamental Escherichia coli biochemical pathways for biomass and energy production: creation of overall flux states. Biotechnology & Bioengineering 86(2):149-62.
10. Farmer, W. R., J. C. Liao. 2001. Precursor balancing for metabolic engineering of lycopene production in Escherichia coli. Biotechnol Prog. 17(1):57-61.
11. Ingraham, J. L., O. Maaloe, F. C. Neidhardt. 1983. Growth of the Bacterial Cell. Sunderland, Sinauer Associates Inc.
12. Keseler, I. M., J. Collado-Vides, S. Gama-Castro, J. Ingraham, S. Paley, I. T. Paulsen, M. Peralta-Gil, P. D. Karp. 2005. EcoCyc: a comprehensive database resource for E. coli. Nucleic Acids Res. 33, D334-D337.
13. Lee, P. C., B. N. Mijts, C. Schmidt-Dannert. 2004. Investigation of factors influencing production of the monocyclic carotenoid torulene in metabolically engineered Escherichia coli. Appl. Microbiol. Biotechnol. 65(5):538-46.
14. Mijts, B. N., P. C. Lee, C. Schmidt-Dannert. 2005. Identification of a carotenoid oxygenase synthesizing acyclic xanthophylls combinatorial biosynthesis and directed evolution. Chem. Biol. 12: 453-60.
15. Pfeiffer, T., I. Sanchez-Valdenebro, J. C. Nuno, F. Montero, S. Schuster. 1999. Metatool: For studying metabolic networks. Bioinformatics 15(3): 251-57.
16. Ruther, A., N. Misawa, P. Boger, G. Sandmann. 1997. Production of zeaxanthin in Escherichia coli transformed with different carotenogenic plasmids. Appl Microbiol Biotechnol. 48: 162-67.
17. Schuster, S., D. Fell, T. Dandekar. 2000. A general definition of metabolic pathways useful for systematic organization and analysis of complex metabolic networks. Nature Biotechnology 18:326-32.
18. Sprenger, G. 1995. Genetics of pentose-phosphate pathway enzymes of Escherichia coli K-12. Arch Microbiol. 164: 324-30.
19. Stelling, J., S. Klamt, K. Bettenbrock, S. Schuster, E. D. Gilles. 2002. Metabolic network structure determines key aspects of functionality and regulation. Nature 420: 190-93.
20. Tao, L., A. Schenzle, J. M. Odom and Q. Cheng. 2005. Novel carotenoid oxidase involved in biosynthesis of 4,4′-diapolycopene dialdehyde. Appl Environ Microbiol. 71: 3294-301.
21. Trinh, C. T., R. Carlson, A. Wlaschin, F. Srienc. 2006. Design, construction and performance of the most efficient biomass producing E. coli bacterium. Metab Eng. 8(6):628-38.
22. Trinh, C. T., P. Unrean, F. Srienc. 2008. Minimal Escherichia coli cell for the most efficient production of ethanol from hexoses and pentoses. Appl Environ Microbiol. 74(12):3634-43.
23. Vershinin, A. 1999. Biological functions of carotenoids—diversity and evolution. Biofactors 10:99-104
24. Wang, C. W., M. K. Oh, J. C. Liao. 1998. Engineered isoprenoid pathway enhances Astaxanthin production in Escherichia coli. Biotechnol Bioeng. 62(2):235-41.
25. Wieland, B., C. Feil, E. Gloria-Maercker, G. Thumm, M. Lechner, J. M. Bravo, K. Poralla, F. Götz. 1994. Genetic and Biochemical Analyses of the Biosynthesis of the Yellow Carotenoid 4,4′-Diaponeurosporene of Staphylococcus aureus. J. Bacteriol. 176(24): 7719-26.

TABLE 1
Strains used in this study

| | Derived from | | |
|---|---|---|---|
| Strain | parental strain | Modifications | References |
| Wild-type | MG1655 | None | Bachmann 1996 |
| JW1375 | BW25113 | ΔldhA::kan+ | Baba et al 2006 |
| JW4115 | BW25113 | ΔfrdA::kan+ | Baba et al 2006 |
| JW0855 | BW25113 | ΔpoxB::kan+ | Baba et al 2006 |
| JW2294 | BW2229 | Δpta::kan+ | Baba et al 2006 |
| JW1228 | BW25113 | ΔadhE::kan+ | Baba et al 2006 |
| JW1666 | BW25113 | ΔpykF::kan+ | Baba et al 2006 |
| JW1841 | BW25113 | Δzwf::kan+ | Baba et al 2006 |
| JW2447 | BW25113 | ΔmaeB::kan+ | Baba et al 2006 |
| CRT028 | MG1655 | ΔldhAΔfrdAΔpoxBΔptaΔadhEΔpykF | This study |
| | | ΔzwfΔmaeB::kan− | |

TABLE 2
Primer sequences designed for testing specific deleted genes

| Deleted | | | |
|---|---|---|---|
| genes | Left primer | Right primer | Position(1) |
| IdhA | 5′-CGGCTTTATATTTACCCAGC-3′ | 5′-CGCAACAAACGCGGCTAC-3′ | Outside |
| | (SEQ ID NO:1) | (SEQ ID NO:2) | |
| frdA | 5′-ACGCTTCAACCTTCATACCG-3′ | 5′-GATGCCGTTTCGCTCATAGT-3′ | Outside |
| | (SEQ ID NO:3) | (SEQ ID NO:4) | |
| poxB | 5′-ATGGATATCGTCGGGTTTGA-3′ | 5′-AAGCAATAACGTTCCGGTTG-3′ | Outside |
| | (SEQ ID NO:5) | (SEQ ID NO:6) | |
| pta | 5′-GTTCGCCTGCTTCGTTAGTC-3′ | 5′-CTGCCGCTATGTTGAAGACA-3′ | Outside |
| | (SEQ ID NO:7) | (SEQ ID NO:8) | |
| adhE | 5′-AAAGCGATGCTGAAAGGTGT-3′ | 5′-AGAAAGCGTCAGGCAGTGTT-3′ | Outside |
| | (SEQ ID NO:9) | (SEQ ID NO:10) | |
| pykF | 5′-GCGCCAATTGACTCTTGAAT-3′ | 5′-CCTGCCAGCAGAGTAGAACC-3′ | Outside |
| | (SEQ ID NO:11) | (SEQ ID NO:12) | |
| zwf | 5′-CGCGTAACAATTGTGG-3′ | 5′-CTGGATTTTTTCCAGC-3′ | Outside |
| | (SEQ ID NO:13) | (SEQ ID NO:14) | |
| maeB | 5′-CTGTTTGATGCCGTCTAACTCGTTC-3′ | 5′-CTTTATCCATGAGTCGCCGCCTGTG-3′ | Outside |
| | (SEQ ID NO:15) | (SEQ ID NO:16) | |
| IdhA | 5′-TACCCAACGAACCAATTTTC-3′ | 5′-GCTGGAAGAGCTGAAAAAGC-3′ | Inside |
| | (SEQ ID NO:17) | (SEQ ID NO:18) | |
| frdA | 5′-TTACGTGCCATTGCGGAGTG-3′ | 5′-TCACGATACAGTAGCGGGTG-3′ | Inside |
| | (SEQ ID NO:19) | (SEQ ID NO:20) | |
| poxB | 5′-CCACCAGCTTTCATCTCCAT-3′ | 5′-TATTCCCTCCAGCGAAATTG-3′ | Inside |
| | (SEQ ID NO:21) | (SEQ ID NO:22) | |
| pta | 5′-TCAGATCCGGGAAGATGAAC-3′ | 5′-TGTGCTGATGGAAGAGATCG-3′ | Inside |
| | (SEQ ID NO:23) | (SEQ ID NO:24) | |
| adhE | 5′-TGAATGCAGTCTGCTTGGTC-3′ | 5′-AAAACGTTGGTCCGAAACAC-3′ | Inside |
| | (SEQ ID NO:25) | (SEQ ID NO:26) | |
| pykF | 5′-CACCGTACTGGTTGACGATG-3′ | 5′-CACAACGCCTTTGCTCAGTA-3′ | Inside |
| | (SEQ ID NO:27) | (SEQ ID NO:28) | |
| zwf | 5′-TCTACCCATTTCCAGGCTTC-3′ | 5′-TGGGACACCCTGAGTGCACG-3′ | Inside |
| | (SEQ ID NO:29) | (SEQ ID NO:30) | |
| maeB | 5′-GAGCTGTCCGGCATACGGTC-3′ | 5′-CGCTGGCAGGCAAACCGGTG-3′ | Inside |
| | (SEQ ID NO:31) | (SEQ ID NO:32) | |

(1) Inside means the primers specified to deleted part of the gene, while outside means the primers specified to undeleted portions of the gene.

TABLE 3
Total remaining elementary modes after sequential deletion of multiple genes.

| | | | | Predicted |
|---|---|---|---|---|
| | | Aerobic | Anaerobic | CRT |
| Strain | Total modes | modes | modes | Yield(1) |
| Wild-type | 29,532 | 24,155 | 5,377 | 0.0-426 |
| ΔldhA | 15,662 | 13,405 | 2,257 | 0.0-426 |
| ΔldhAΔfrdA | 8,573 | 7,810 | 763 | 0.0-426 |
| ΔldhAΔfrdAΔpoxB | 7,541 | 6,861 | 680 | 0.0-426 |
| ΔldhAΔfrdAΔpoxBΔpta | 6,171 | 5,600 | 571 | 0.0-426 |
| ΔldhAΔfrdAΔpoxBΔptaΔa dhE | 4,099 | 4,099 | 0 | 0.0-426 |
| ΔldhAΔfrdAΔpoxBΔptaΔadhEΔpykF | 2,573 | 2,573 | 0 | 0.0-426 |
| ΔldhAΔfrdAΔpoxBΔptaΔadhEΔpykFΔzwf | 375 | 375 | 0 | 0.0-426 |
| ΔldhAΔfrdAΔpoxBΔptaΔadhEΔpykFΔzwfΔmaeB | 5 | 5 | 0 | 0.4-426 |

The table illustrates the reduction of elementary modes after each sequential gene deletion. The progressively decreasing numbers of available elementary modes after multiple gene deletions limits the cell's pathway options, hence forcing the cell to operate using only the efficient one. Elementary modes are categorized as aerobic modes (oxygen consuming modes) and anaerobic modes which do not use oxygen.
(1)Yield is in mg-diapolycopendioic acid/g-glucose

TABLE 4
Gene knockout targets for improving the production
of diapolycopendioic acid in recombinant E. coli.

| Deleted | Corresponding | | |
|---|---|---|---|
| Reaction | gene | Enzyme | Pathway |
| R9 | pykF | Pyruvate kinase | Glycolysis |
| R11 | zwf | Glucose-6-phosphate-1- | Pentose |
| | | dehydrogenase | phosphate |
| R22 | frdA | Fumarate reductase | Fermentation |
| R28 | maeB | Malate dehydrogenase | Anapleurotic |
| R31 | poxB | Pyruvate oxidase | Fermentation |
| R32 | ldhA | Lactate dehydrogenase | Fermentation |
| R34 | adhE | Alcohol dehydrogenase | Fermentation |
| R35 | pta | Phosphate acetyltransferase | Fermentation |

Gene and enzyme annotation were obtained from Ecocyc database at www.ecocyc.org

TABLE 5
Overall reaction stoichiometry and product yields
of each elementary modes remained in the multiple-genes
knockout, CRT028/pACMNOx.

| EMs | Overall reaction stoichiometry | Y_{P}¹ | Y_{X}¹ |
|---|---|---|---|
| 1 | Glucose + 0.14 O₂ → 0.17 Carotenoids + 1 CO₂ | 426 | 0 |
| 2 | Glucose + 0.14 O₂ → 0.17 Carotenoids + 1 CO₂ | 426 | 0 |
| 3 | Glucose + 0.14 O₂ → 0.17 Carotenoids + 1 CO₂ | 426 | 0 |
| 4 | Glucose + 0.11 O₂ + 0.53 NH₃ → 0.11 | 270 | 0.26 |
| | Carotenoid + 0.91 CO₂ + 6.96 × 10⁻⁴ | | |
| | Biomass | | |
| 5 | Glucose + 0.22 O₂ + 1.30 NH₃ → 1.56 × | 0.41 | 0.64 |
| | 10⁻⁴ Carotenoid + 1 CO₂ + 1.73 × | | |
| | 10⁻³ Biomass | | |

Carotenoid yield is presented in mg carotenoid/g glucose while biomass yield is shown in g biomass/g glucose
¹Y_{P} is carotenoid yield while biomass yield is shown in Y_{X}

TABLE 6
Comparison of wild-type MG1655/pACMNOx and multiple-
genes knockout CRT028/pACMNOx performance on the
synthesis of carotenoids, diapolycopendial and
diapolycopendioic acid in aerobic batch bioreactor

| | MG1655/pACMNOx | CRT028/pACMNOx |
|---|---|---|
| Growth rate | 0.17 ± 0.02 | 0.13 ± 0.01 |
| hr-1 | | |
| Carotenoid production | 0.19 ± 0.02 | 0.83 ± 0.20 |
| mg/l | | |
| Carotenoid yield | 0.04 ± 0.00 | 0.17 ± 0.04 |
| mg carotenoid/g | | |
| glucose | | |
| Specific production | 0.01 ± 0.00 | 0.10 ± 0.02 |
| mg carotenoid/g cell | | |
| dry weight-hr | | |

TABLE 7
Elementary mode analysis of knockout E. coli strains using
glucose as a carbon source under aerobic conditions for production
of diapolycopendioic acid

| | | | | CRT yield of | |
|---|---|---|---|---|---|
| | | CRT yield | CRT- | CRT- | |
| Knockout/pACMN | | of total | producing | producing | |
| Ox | EMs | Total EMs | EMs | EMs | References |
| ΔpykFΔpykA | 4,275 | 0.00-426 | 929 | <0.01-426 | Farmer |
| | | | | | and Liao |
| | | | | | 2001 |
| ΔaceEΔfdhF | 1,049 | 0.00-426 | 242 | 0.10-426 | Alper et |
| | | | | | al 2005 |
| ΔaceEΔtalB | 952 | 0.00-426 | 352 | 0.02-426 | Alper et |
| | | | | | al 2005 |
| ΔaceEΔtalBΔfdhF | 303 | 0.00-426 | 114 | 0.10-426 | Alper et |
| | | | | | al 2005 |
| ΔldhAΔfrdAΔadhE | 5 | 0.41-426 | 5 | 0.41-426 | This |
| ΔpoxB | | | | | study |
| ΔptaΔpykFΔzwfΔmaeB | | | | | |

CRT, carotenoid diapolycopendioic acid. Yield is in mg-carotenoid/g-glucose.

1. An E. coli for carotenoid production comprising:
exogenous nucleic acids for expressing a carotenoid and diminished or abrogated expression of a gene in the group consisting of ldhA, frdA, poxB, pta, adhE, pykf, zwf, and maeB.

2. The E. coli of claim 1 lacking the genes ldhA, frdA, poxB, pta, adhE, pykf, zwf, and maeB.

3. The E. coli of claim 1 having diminished or abrogated expression of at least five of the genes ldhA, frdA, poxB, pta, adhE, pykf, zwf, and maeB.

4. The E. coli of claim 1 having diminished or abrogated expression of zwf and/or maeB.

5. The E. coli of claim 1 wherein the carotenoid comprises diapolycopendial (DPL) and diapolycopendioic acid (DPA).

6. The E. coli of claim 1 wherein the exogenous nucleic acids for expressing a carotenoid comprise crtM, crtN and crtOx.

7. An engineered E. coli for carotenoid production comprising:
exogenous nucleic acids for expressing a carotenoid that the E. coli requires as necessary condition for the E. coli to reproduce.

8. The E. coli of claim 7 wherein all of the E coli metabolic pathways that receive glucose as an input also result in production of the carotenoid.

9. The E. coli of claim 7 wherein the E. coli having diminished or abrogated expression of a gene in the group consisting of ldhA, frdA, poxB, pta, adhE, pykF, zwf, and maeB.

10. The E. coli of claim 7 wherein the carotenoid comprises diapolycopendial (DPL) and diapolycopendioic acid (DPA).

11. A method of producing a carotenoid comprising culturing a collection of the E. coli of claim 1 and separating the carotenoid from the culture.

12. The method of claim 11 wherein the separation of the carotenoid provides a yield of at least 0.15 mg-carotenoid/g-glucose.

13. A method of producing a carotenoid comprising culturing a collection of the E. coli of claim 7 and separating the carotenoid from the culture.

14. A method of making an improved E. coli comprising modifying a parent E. coli to have diminished or abrogated expression of a gene in the group consisting of ldhA, frdA, poxB, pta, adhE, pykf, zwf, and maeB.

15. The method of claim 14 comprising deleting the genes ldhA, frdA, poxB, pta, adhE, pykf, zwf, and maeB.

16. The method of claim 14 wherein the improved E. coli provides a yield at least about 200% greater than the parent E. coli.

17. The method of claim 14 wherein the improved E. coli elementary modes consist of carotenoid-producing elementary modes.
